(19) United States
(12) Patent Application Publication
(10) Pub. No.: US 2026/0069335 A1
(43) Pub. Date: Mar. 12, 2026

(54) DEVICE FOR MIXING AND INJECTING A COMPOSITION, IN PARTICULAR A BONE CEMENT

(71) Applicant: HISTONE, TOULOUSE (FR)

(72) Inventors: Elena Julia NAVARRO NIXON, TOULOUSE (FR); Nourédine SAHRAOUI, TOULOUSE (FR)

(21) Appl. No.: 19/275,927

(22) Filed: Jul. 21, 2025

(30) Foreign Application Priority Data
Jul. 25, 2024 (FR) 2408243

(51) Int. Cl. A61B 17/88

(52) U.S. Cl. CPC A61B 17/8833; A61B 17/8822; A61B 2017/8838

(57) ABSTRACT

A device to mix and inject a composition includes a vessel, a piston assembly, and a mixing assembly. The vessel delimits an inner chamber and has a tapped opening. The piston assembly includes a threaded rod, a handle and a piston head. The threaded rod cooperates with the tapped opening of the vessel and includes a longitudinal internal through-channel. The piston head is arranged in the inner chamber and includes a through-hole in the continuation of the longitudinal internal through-channel. The mixing assembly includes a rod, a handle and a mixing paddle. The rod extends into the longitudinal internal through-channel of the threaded rod and into the through-hole of the piston head. The mixing paddle is arranged in the inner chamber.

### RELATED APPLICATION

This application claims priority from French Patent Application No. 2408243 filed Jul. 25, 2024, which is incorporated herein by reference in its entirety.

### TECHNICAL FIELD OF THE INVENTION

The present invention relates to a device for mixing and injecting a composition. The subject matter of the invention has a particularly advantageous application in the field of bone surgery. The composition is a bone cement, particularly of the type to be used for bone reconstruction.

### BACKGROUND OF THE INVENTION

In many fields, it may be necessary for components, of the same or different phases, to be mixed shortly before use, for example because the obtained composition is unstable or because the obtained composition solidifies in a relatively short period of time.

In the building sector, for example, it is known to inject a composition into cracks in the walls of buildings in order to fill them. The composition, of the adhesive type, is typically fast-setting.

In the medical field, more particularly, it is known to inject a composition, commonly referred to as bone cement, into a bone to be treated in order to consolidate it. Surgical interventions resorting thereto, such as cementoplasty, vertebroplasty and arthroplasty, for example, allow treating trauma, such as bone fractures, or disease-related bone damage. It is also known to inject bone cement for example in order to place implants, such as hip joints.

Conventionally used bone cements consist of two components, typically a powder and a liquid, which are mixed to obtain a homogeneous composition, in the form of a relatively thick paste. Bone cements are also fast-setting and typically set in around fifteen minutes. It is therefore important to mix the components and inject the bone cement into the area to be treated in a relatively short time.

Some devices have been developed, some of which to enable mixing of the components making up the bone cement, and others to enable the injection of the obtained bone cement into the bone to be treated.

However, these devices have many drawbacks. Some devices prove to be of complex construction, in particular because they are composed of many parts to be assembled, and their use consequently requires a lot of handling by an operator. Other devices are large and/or have a significant weight, making them difficult for the operator to operate. Moreover, the operator must operate several devices, one device for mixing the components and one device for injecting the bone cement. Handling multiple devices in succession leads to a significant loss of time, and yet the bone cement is fast setting. Furthermore, the surgical operating time, and therefore the time under anesthesia, is extended accordingly. Storage and recycling of these devices also pose a significant problem.

Finally, not all devices commercially available are suitable for bone cements of the pasty or very pasty type, i.e. those having a high viscosity. High viscosity is understood to mean a viscosity of at least 80 MPa. s. Devices for mixing the components are, in some cases, incapable of mixing components in order to obtain such a bone cement, whereas devices for injecting the bone cement are, in many cases, incapable of injecting such a very pasty bone cement.

### OBJECT AND SUMMARY OF THE INVENTION

The present invention aims to overcome the aforementioned drawbacks.

To this end, the present invention relates to a device for mixing and injecting a composition, said device comprising:
a vessel comprising a cylindrical wall, a first end and a second end, defining an inner chamber, and having a longitudinal axis, the vessel comprising, at its first end, a tapped opening, the vessel comprising, at its second end, an outlet,
a piston assembly comprising a threaded rod, a handle located at a first end of the threaded rod and a piston head located at a second end of the threaded rod,
   the threaded rod:
      cooperating with the tapped opening of the vessel,
      extending along the longitudinal axis of the vessel,
      comprising a longitudinal internal channel opening out at its two longitudinal ends,
   the piston head:
      being arranged in the inner chamber of the vessel,
      having a cross-section whose shape is complementary to that of the cross-section of the cylindrical wall of the vessel,
      comprising a through-hole in the continuation of the longitudinal internal channel of the threaded rod,
a mixing assembly comprising a rod, a handle located at a first end of the rod and a mixing paddle located at a second end of the rod, the rod of the mixing assembly extending into the longitudinal internal channel of the threaded rod and into the through-hole of the piston head of the piston assembly,
   the mixing paddle being arranged in the inner chamber of the vessel.

The mixing assembly is intended to allow an operator to mix the components previously introduced into the inner chamber of the vessel in order to obtain the composition. The piston assembly is intended to allow the operator to transfer the composition obtained inside the inner chamber of the vessel, after the mixing of the components by the mixing assembly, out of said inner chamber, via the outlet.

The mixing assembly is used during a so-called mixing phase. The piston assembly is used during a so-called injection phase.

During the mixing phase, the piston head of the piston assembly is placed at the first end of the vessel. The operator only operates the handle of the mixing assembly by imparting, in particular, a translational motion to the mixing assembly. This translational motion enables the translation displacement of the mixing paddle in the inner chamber of the vessel, between the first end and the second end of said vessel. Thus, the displacement of the mixing paddle in the inner chamber of the vessel enables mixing of the components together until the desired composition is obtained.

During the injection phase, the operator only operates the handle of the piston assembly and rotates it to screw the threaded rod into the tapped opening of the vessel, causing the piston head to translate towards the second end of the vessel, pushing the obtained composition through the outlet of the vessel.

Thus, the device according to the invention advantageously enables both mixing of the components making up the composition, via the mixing assembly, and injection of the composition, via the piston assembly.

Because the mixing assembly is nested in the piston assembly, the device according to the invention proves to be small in volume, in particular with regards to storage thereof before use.

Due to the few parts that make it up, the device according to the invention requires less handling by the operator and is easy to use. This saves the operator time compared to when using existing devices, and therefore allows for the use of fast-setting cements.

Thanks to the engagement of the threaded rod of the piston assembly in the tapped opening of the vessel, the device according to the invention advantageously allows easily injecting the composition, even when it is pasty, i.e. a composition having a high viscosity, without breaking.

The composition can be obtained, without limitation, from the mixture of:
a powder and a liquid, or
two liquids,
two gels,
a gel and a liquid.

The device according to the invention can be used in many fields of application, such as, for example in the construction sector. Thus, the device enables mixing and injection of a composition into cracks at the walls of buildings in order to fill them.

The device according to the invention is particularly suitable for use in a medical environment, in particular for bone surgery, such as cementoplasty, vertebroplasty, arthroplasty, in order to consolidate a bone or a vertebra. The fact that the device can mix and inject a pasty-type bone cement is advantageous in that the surgical operating time, and therefore the time under anesthesia, is reduced, thereby reducing the risk to patients. In addition, the use of a pasty bone cement reduces the risk of the bone cement leaking into the body of the patient.

According to particular embodiments, the invention also has the following features, which can be implemented separately or according to any technically feasible combination thereof.

In particular embodiments, to enable the introduction of the components into the inner chamber of the vessel, the vessel is made into two removable parts, a first part comprising the tapped opening of the vessel and a second part comprising the outlet of the vessel, the first part forming a cover for the second part.

In particular embodiments, to enable the introduction of the components making up the composition into the inner chamber of the vessel, the vessel comprises a hole made in the cylindrical wall.

In particular embodiments, the rod of the mixing assembly is an elongate body, smooth in appearance on the outside, and has a cross-section whose shape complements that of the cross-section of the longitudinal internal channel of the threaded rod of the piston assembly. The handle of the mixing assembly is rigidly connected to the rod of the mixing assembly. The operator should then impart a translational motion, along the longitudinal axis, and a rotational motion, about the longitudinal axis, to the handle of the mixing assembly in order to translate and rotate the mixing paddle.

The term “connected” is understood to mean that the parts are linked together by a connection enabling a relative motion of one part relative to the other. The term “rigidly connected” is understood to mean that the parts are connected together in a fixed manner, i.e. any relative motion therebetween is not possible.

In particular embodiments, the longitudinal internal channel of the threaded rod of the piston assembly has an inner helical rib. The rod of the mixing assembly is an elongated body, having an outer helical groove, said outer helical groove cooperating with the inner helical rib of the longitudinal internal channel of the threaded rod of the piston assembly. The handle of the mixing assembly is connected to the rod of the mixing assembly. The operator then needs to impart only a translational motion, along the longitudinal axis, to the handle of the mixing assembly in order to translate and rotate the mixing paddle of the mixing assembly. The engagement of the helical groove of the rod in the helical rib of the longitudinal internal channel of the threaded rod translates and rotates said rod, and consequently translates and rotates the mixing paddle in the vessel.

In particular embodiments, the rod of the mixing assembly is an elongate body having an outer helical groove. The longitudinal internal channel of the threaded rod of the piston assembly has a smooth appearance and has a cross-section larger than the cross-section of the rod of the mixing assembly. The through-cavity of the handle of the piston assembly has a cross-section smaller than the cross-section of the longitudinal internal channel of the threaded rod of the piston assembly and has an inner helical rib, the outer helical groove cooperating with the inner helical rib of the through-cavity of the handle of the piston assembly. The handle of the mixing assembly is connected to the rod of the mixing assembly. The operator then needs to impart only a translational motion, along the longitudinal axis, to the handle of the mixing assembly in order to translate and rotate the mixing paddle of the mixing assembly. The engagement of the outer helical groove of the rod of the mixing assembly in the inner helical rib of the through-cavity of the handle of the piston assembly translates and rotates said rod of the mixing assembly, and consequently translates and rotates the mixing paddle in the vessel.

In particular embodiments, to reduce repetitive back-and-forth movements of the mixing assembly for the operator, the device comprises a resilient return member, for example a spring, configured to return the mixing assembly to an initial position in which the mixing paddle of the mixing assembly is against the piston head of the piston assembly.

In particular embodiments, to prevent the mixing paddle from moving in the inner chamber of the vessel during the injection phase, the mixing paddle is rigidly connected to the piston head, during said injection phase.

In particular embodiments, in order to rigidly connect the mixing paddle to the piston head, the mixing paddle of the mixing assembly comprises a sleeve, and the piston head of the piston assembly comprises a recess suitable for receiving the sleeve by interlocking therewith.

In particular embodiments, to prevent the rod of the mixing assembly from obstructing movements of the operator during the injection phase, the rod of the mixing assembly is removable.

In particular example embodiments, the rod of the mixing assembly comprises, at its second longitudinal end, a thread. The sleeve of the mixing paddle comprises a tapped central cavity. The thread of the rod and the tapping of the central cavity cooperate with one another. Thus, the rod of the mixing assembly can be unscrewed from the mixing paddle and removed from the piston assembly and thus from the device.

In particular embodiments, to avoid the rod of the mixing assembly obstructing the movements of the operator during the injection phase, the rod of the mixing assembly has a weak zone allowing initiating the break-up of said rod.

In particular embodiments, to facilitate gripping of the device by the operator, the device comprises a gripping member, of the handle type. In particular example embodiments, said gripping member can be formed by two half-shells assembled together, surrounding the vessel at its cylindrical wall. The first and second ends of the vessel are not enclosed by the two half-shells.

In particular example embodiments, said gripping member can be formed by two half-shells assembled to each other, surrounding the vessel, at least at its first end, and a portion of the threaded rod of the piston assembly.

### BRIEF DESCRIPTION OF THE FIGURES

The invention will be better understood upon reading the following description, which is given as a non-limiting example, and made with reference to the following figures:

FIG. 1 illustrates a sectional view of an example device for mixing and injecting a composition according to the invention;

FIG. 2 shows two alternative embodiments of an example vessel of the device for mixing and injecting a composition;

FIG. 3 shows two further example vessels of the device for mixing and injecting a composition;

FIG. 4 shows a perspective view of a part of a piston assembly of the device for mixing and injecting a composition;

FIG. 5 illustrates a perspective view of an example of a piston head of the piston assembly of the device for mixing and injecting a composition;

FIG. 6 is a front view of the piston head of the piston assembly of the device for mixing and injecting a composition;

FIG. 7 is a perspective view of an example of a mixing assembly of the device for mixing and injecting a composition;

FIG. 8 is a perspective view of a part of a mixing assembly of the device for mixing and injecting a composition;

FIG. 9 shows a sectional view of the device for mixing and injecting a composition of FIG. 1, during a mixing phase;

FIG. 10 shows a sectional view of the device for mixing and injecting a composition of FIG. 1, before an injection phase;

FIG. 11 shows a sectional view of the device for mixing and injecting a composition of FIG. 1, during the injection phase;

FIG. 12 shows a sectional view of an alternative embodiment of the device for mixing and injecting a composition.

In these figures, identical reference numerals from one figure to another refer to identical or similar elements. Moreover, for reasons of clarity, the drawings are not to scale unless specified otherwise.

### DETAILED DESCRIPTION OF THE EMBODIMENTS

The invention relates to a device for mixing and injecting a composition according to the invention.

In the remainder of the description, the device for mixing and injecting a composition will simply be referred to as the device 100.

The invention is described in the particular context of one of its preferred fields of application, wherein the device 100 is intended to be used in a medical environment, in particular for bone surgery procedures, such as cementoplasty, vertebroplasty, or arthroplasty, in order to consolidate a bone or a vertebra.

The device according to the invention advantageously makes it possible both to receive the constituent components of the desired composition, referred to as the bone cement, to mix said components in order to obtain the bone cement, and to inject the bone cement.

The bone cement used in surgical procedures is generally obtained, without limitation, from a mixture of a powder and a liquid.

In one embodiment, the powder is a polymethyl methacrylate (PMMA) powder and the liquid contains methyl methacrylate (MMA) molecules.

After mixing, the bone cement is typically in the form of a relatively thick paste. The resulting bone cement has appropriate mechanical and chemical properties and is biocompatible.

The preparation of such a bone cement is within the reach of a person skilled in the art.

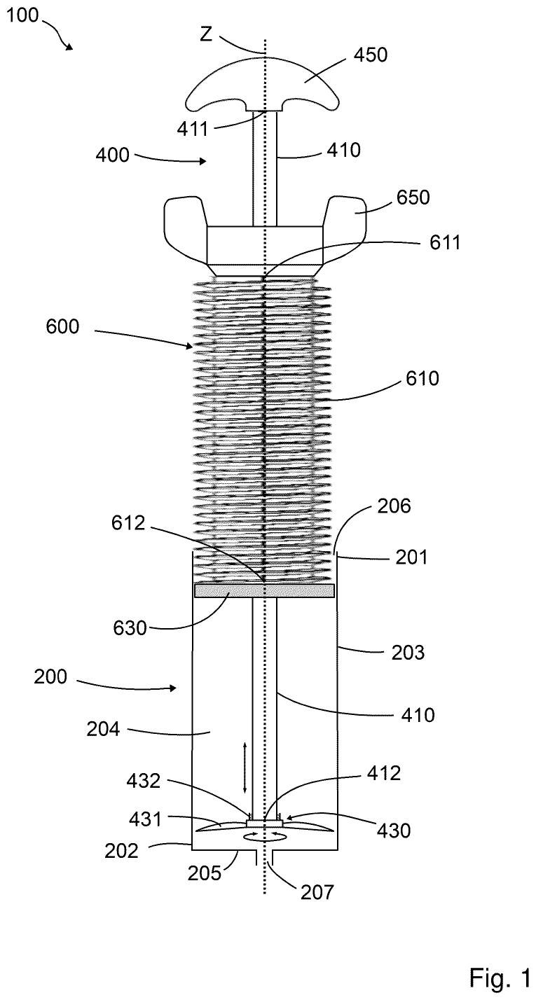

FIG. 1 illustrates an example device 100 according to the invention.

The device 100 comprises a vessel 200.

The vessel 200 has the shape of a hollow cylindrical body, preferably having a circular cross-section. The vessel 200 comprises a cylindrical wall 203, a first end 201, a second end 202, and has a longitudinal axis Z. The vessel 200 defines an inner chamber 204 into which components can be introduced to be mixed therein in order to form the bone cement.

The vessel 200 comprises, at its second end 202, an outlet 207. The outlet 207 is also in communication with the inner chamber 204 of the vessel 200. Preferably, the vessel 200 has, at its second end 202, a wall 205 comprising the outlet 207, as illustrated in FIG. 1.

The outlet 207 may be equipped with a connector (not shown) capable of cooperating with a complementary connector of another medical device, such as a trocar, a cannula, a needle, in order to transfer the bone cement into the area to be treated. The connector can, for example, be of the Luer Lock type.

The outlet 207 may be equipped with a cover (not shown) to prevent the bone cement from flowing through the outlet 207 during mixing of the components in the inner chamber 204 of the vessel 200.

The vessel 200 comprises, at its first end 201, a tapped opening 206. The tapping of the opening is an internal thread, i.e. it is made inside the opening. The tapping of the opening is not shown in FIG. 1, and 9-11.

The tapped opening 206 is in communication with the inner chamber 204 of the vessel 200. The tapped opening 206 is centered about the longitudinal axis Z of the vessel 200.

The tapped opening 206 can form all or part of the first end 201 of the vessel 200. In the example of view a) of FIG. 3, the vessel 200 has, at its first end 201, a wall comprising the tapped opening 206. In the example of view b) of FIG. 3, the tapped opening 206 is formed over the entire first end 201 of the vessel 200. The opening is then tapped at an inner surface 209 of the cylindrical wall 203 of the vessel 200.

The vessel 200 can be made in one piece, as illustrated in views a) and b) of FIG. 3. Preferably, the vessel 200 comprises a hole 208 made in the cylindrical wall 203 of the vessel 200, for introducing the components into the inner chamber 204 of the vessel 200. Said hole 208 can be equipped with a connector (not shown) capable of cooperating with a complementary connector of another medical device, such as a syringe or a funnel, for transferring the components constituting the bone cement into the inner chamber 204 of the vessel 200. The connector can, for example, be of the Luer-Lock type.

The hole 208 may be equipped with a cover (not shown) to prevent the bone cement from flowing through the hole 208 during mixing of the components in the inner chamber 204 of the vessel 200.

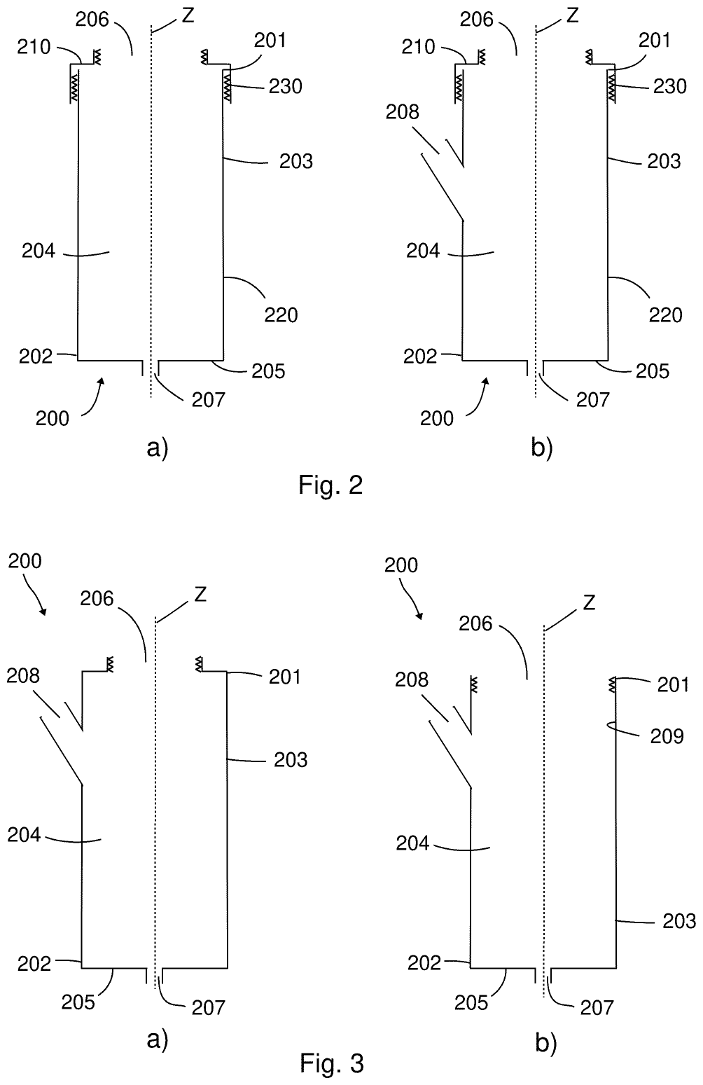

The vessel 200 may be made into two removable parts 210, 220, as illustrated in view a) of FIG. 2, to enable the introduction of the components into the inner chamber 204 of the vessel 200. For example, a first part 210 can comprise the tapped opening 206 of the vessel and a second part 220 can comprise the outlet 207 of the vessel. The first part 210 can, for example, form a cover for the second part 220. The two parts 210, 220 are assembled together by reversible connection means, for example by a threading-tapping type connection 230. The first part 210, respectively the second part 220, comprises a thread, respectively a tapping, or vice versa, the thread and the tapping cooperating so that the two parts 210, 220 can be assembled or disassembled.

The vessel 200, made of two removable parts 210, 220, can further comprise the hole 208 made in the cylindrical wall 203 of the vessel 200, for introducing the components into the inner chamber 204 of the vessel 200, as illustrated in view b) of FIG. 3. The second part 220 can, for example, comprise the hole 208. Said hole 208 can be equipped with a connector (not shown) capable of cooperating with a complementary connector of another medical device, such as a syringe or a funnel, for transferring the components constituting the bone cement into the inner chamber 204 of the vessel 200. The connector can, for example, be of the Luer-Lock type.

The hole 208 may be equipped with a cover (not shown) to prevent the bone cement from flowing through the hole 208 during mixing of the components in the inner chamber 204 of the vessel 200.

The vessel 200 can be made of a transparent material, for example glass or polycarbonate, to allow an operator to view the contents of the vessel 200.

The device 100 further comprises a mixing assembly 400 and a piston assembly 600.

The purpose of the mixing assembly 400 is to allow the operator to mix the components previously introduced into the inner chamber 204 of the vessel 200 in order to obtain the bone cement. The mixing assembly 400 of the device 100 is intended to be used during a phase called mixing phase, which will be described hereinafter.

In turn, the piston assembly 600 is intended to enable the operator to transfer the obtained bone cement in the inner chamber 204 of the vessel 200 out of said inner chamber 204, via the outlet 207. The piston assembly 600 of the device 100 is intended to be used during a phase called injection phase, which will be described hereinafter. It is clear from the description that the injection phase is carried out after the mixing phase.

The piston assembly 600 comprises a threaded rod 610, a piston head 630, and a handle 650, as shown in FIG. 1 and 4-6.

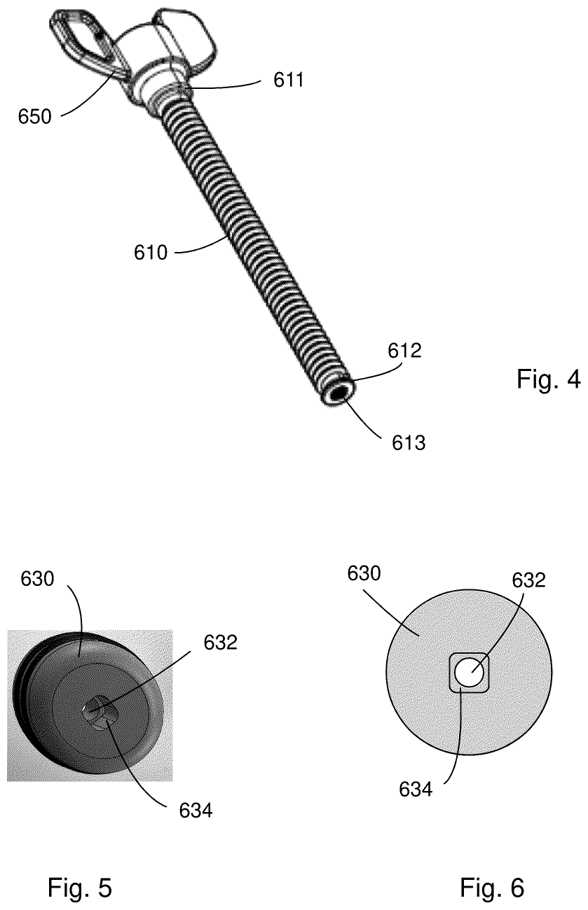

The thread of the threaded rod 610 is an external thread, i.e. a thread made on an external surface of the threaded rod 610, as illustrated in FIG. 4. The threaded rod 610 cooperates with the tapped opening 206 of the vessel 200. The threaded rod 610 thus has a longitudinal axis coaxial with the longitudinal axis Z of the vessel 200.

The threaded rod 610 extends between two longitudinal ends 611, 612. A first longitudinal end 611 is intended to move out of the inner chamber 204 of the vessel 200 and a second longitudinal end 612 is intended to move within said inner chamber 204.

The threaded rod 610 is a hollow threaded rod 610, i.e. it comprises a longitudinal internal channel 613 opening out at its two longitudinal ends 611, 612.

The handle 650 of the piston assembly 600 comprises a through-cavity (not shown in the figures) in the extension of the longitudinal internal channel 613.

The piston head 630 of the piston assembly 600 is arranged at the second longitudinal end 612 of the threaded rod 610. The piston head 630 is arranged in the inner chamber 204 of the vessel 200 and is configured to be able to translate, along the longitudinal axis Z of the vessel 200, between the first end 201 and the second end 202 of the vessel 200.

The threaded rod 610 thus has a length that is at least sufficient for the piston head 630 to move between the two ends 201, 202 of the vessel 200.

The piston head 630 is rigidly connected to the threaded rod 610.

In the present description, “connected” shall be understood to mean parts connected to each other by a connection enabling a relative motion of one part relative to the other. “Rigidly connected” shall be understood to mean parts that are fixedly connected together, i.e. any relative motion therebetween is not possible.

The piston head 630 is in the form of a solid part. It has a cross-section whose shape complements that of the cross-section of the cylindrical wall 203 of the vessel 200. Thus, when the cylindrical wall 203 of the vessel 200 has a circular cross-section, the piston head 630 is disk-shaped with a diameter substantially equal to the inner diameter of the cylindrical wall 203 of the vessel 200.

A seal (not shown) can, for example, be placed at a periphery of the piston head 630 in order to fill the space between said periphery of the piston head 630 and the cylindrical wall 203 of the vessel 200 and thus ensure sealing between the piston head 630 and the cylindrical wall 203 of the vessel 200.

The piston head 630 has a through-hole 632 (FIGS. 5 and 6) arranged in the continuity of the longitudinal internal channel 613 of the threaded rod 610.

The handle 650 of the piston assembly 600 is arranged at the first longitudinal end 611 of the threaded rod 610 and is intended for the handling of the piston assembly 600 by the operator.

The handle 650 is rigidly connected to the threaded rod 610.

The handle 650 is arranged so as not to obstruct access to the longitudinal internal channel 613 of the threaded rod 610.

To use the piston assembly 600, the operator manipulates the handle 650 of the piston assembly 600 by rotating it about the longitudinal axis Z.

Depending on the direction of rotation of said handle 650, the piston head 630 translates inside the vessel 200, moving away from or towards the first end 201 of said vessel 200.

Thus, when the handle 650 of the piston assembly 600 is operated by the operator in a first direction of rotation, so-called screwing direction, for example in the clockwise direction, the threaded rod 610 is rotated about its longitudinal axis and causes, on the one hand, the translation of the piston head 630 along the longitudinal axis Z towards the second end 202 of the vessel 200 and, on the other hand, the rotation of the piston head about the longitudinal axis Z. When the handle 650 of the piston assembly 600 is operated by the operator in a second direction of rotation, so-called unscrewing direction, i.e. in the counterclockwise direction, the threaded rod 610 is rotated about its longitudinal axis and causes, on the one hand, the translation of the piston head 630 towards the first end 201 of the vessel 200 and, on the other hand, the rotation of the piston head about the longitudinal axis Z.

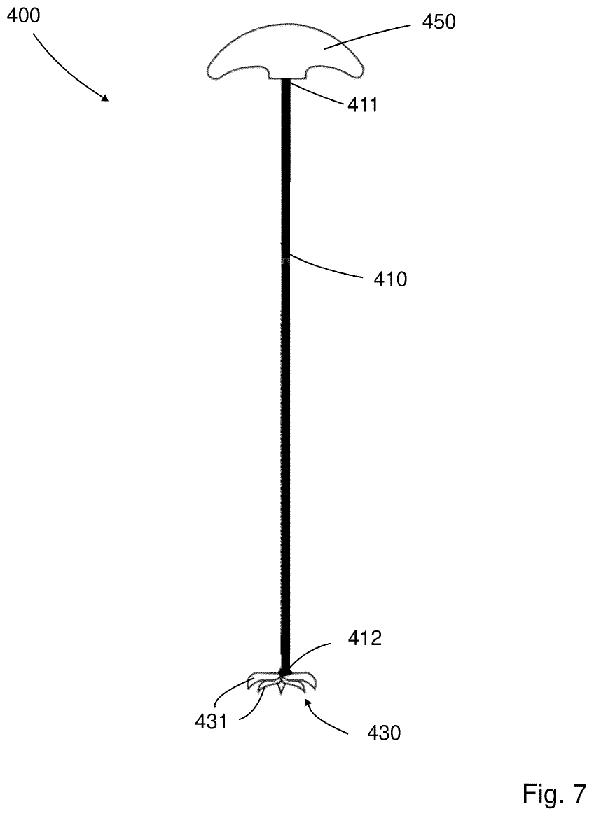

The mixing assembly 400 comprises a rod 410, a mixing paddle 430, and a handle 450, as shown in FIGS. 1 and 7.

The rod 410 passes through the first end 201 of the vessel 200 at the tapped opening 206 of the vessel 200.

The rod 410 extends between two longitudinal ends 411, 412. A first longitudinal end 411 is intended to move out of the inner chamber 204 of the vessel 200 and a second longitudinal end 412 is intended to move within the inner chamber 204 of the vessel 200.

The mixing paddle 430 is arranged at the second longitudinal end 412 of the rod 410. The mixing paddle 430 is arranged in the inner chamber 204 of the vessel 200 and is configured to be movable, on one hand, in translation between the first end 201 and the second end 202 of the vessel 200 and, on the other hand, in rotation about the longitudinal axis Z.

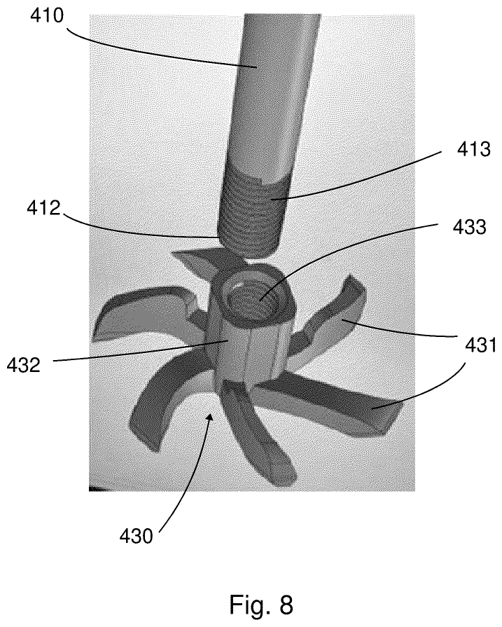

In a preferred example embodiment, as illustrated in FIGS. 1, 7 and 8, the mixing paddle 430 comprises a plurality of blades 431. Each blade 431 extends from the second longitudinal end 412 of the rod 410, toward the cylindrical wall 203 of the vessel 200. In the non-limiting example of FIG. 8, there are six blades 431, each having a curved shape.

In an alternative embodiment (not shown), the mixing paddle 430 is in the form of a perforated disk.

In turn, the handle 450 of the mixing assembly 400 is arranged at the first longitudinal end 411 of the rod 410 and is intended for handling the mixing assembly 400 by the operator.

According to the invention, the mixing assembly 400 is interlocked with the piston assembly 600.

More precisely, the rod 410 of the mixing assembly 400 extends into the longitudinal internal channel 613 of the threaded rod 610 and into the through-hole 632 of the piston head 630 of the piston assembly 600.

Thus, the piston head 630 of the piston assembly 600 is arranged, in the inner chamber 204 of the vessel 200, between the first end 201 of the vessel 200 and the mixing paddle 430 of the mixing assembly 400.

The handle 650 of the piston assembly 600 is arranged, outside the vessel 200, between the first end 201 of the vessel 200 and the handle 450 of the mixing assembly 400.

The rod 410 of the mixing assembly 400 translates along the longitudinal axis Z and rotates about said longitudinal axis within the longitudinal internal channel 613 of the threaded rod 610 of the piston assembly 600 and within the through-hole 632 of the piston head 630 of the piston assembly 600.

Advantageously, the rod 410 of the mixing assembly 400 has a sufficient length so that, when the piston head 630 of the piston assembly 600 is at the first end 201 of the vessel 200, the operator can operate the handle 450 of the mixing assembly 400 so that the mixing paddle 430 of the mixing assembly 400 can move in the inner chamber 204 of the vessel 200, between the piston head 630 and the second end 202 of the vessel 200, as illustrated in FIGS. 1, 9 and 10.

The rod 410 of the mixing assembly 400 thus has a length corresponding at least to the length of the vessel 200 and to the length of the threaded rod 610 of the piston assembly 600.

Thus, upon actuation of the mixing assembly 400, the mixing paddle 430 of the mixing assembly 400 can move within the inner chamber 204 of the vessel 200, between the piston head 630 and the second end 202 of the vessel 200. When the mixing paddle 430 of the mixing assembly 400 is positioned against the piston head 630 of the piston assembly 600, the mixing assembly is in a position called initial position.

In a first version of an embodiment of the device 100, as illustrated in FIG. 1, the rod 410 of the mixing assembly 400 is an elongate body, of smooth appearance on the outside. It has a cross-section whose shape complements that of the cross-section of the longitudinal internal channel 613 of the threaded rod 610 of the piston assembly 600 and of the through-hole 632 of the piston head 630 of the piston assembly 600. Thus, in this first version, the longitudinal internal channel 613 of the threaded rod 610 of the piston assembly 600 makes it possible to guide the translation of the rod 410 of the mixing assembly 400 along the longitudinal axis Z, while allowing it to rotate about said longitudinal axis.

In turn, the handle 450 of the mixing assembly 400 is rigidly connected to the rod 410 of the mixing assembly 400.

In this first version, the operator should impart a translational motion, along the longitudinal axis Z, and a rotational motion, about the longitudinal axis Z, to the handle 450 of the mixing assembly 400 in order to translate and rotate the mixing paddle 430.

In a second version (not shown) of the device 100, the rod 410 of the mixing assembly 400 is an elongate body, having an outer helical groove, i.e. a helical groove formed on an outer surface of the elongate body. The longitudinal internal channel 613 of the threaded rod 610 of the piston assembly 600 has an inner helical rib. The outer helical groove of the rod 410 of the mixing assembly 400 cooperates with the inner helical groove of the longitudinal internal channel 613 of the threaded rod 610 of the piston assembly 600. When the through-cavity in the handle 650 of the piston assembly 600 has a cross-section equal to the cross-section of the longitudinal internal channel 613 of the threaded rod 610 of the piston assembly 600, said through-cavity has an inner helical rib in the extension of the inner helical rib of the longitudinal internal channel 613. When the through-cavity in the handle 650 of the piston assembly 600 has a diameter greater than the diameter of the longitudinal internal channel 613 of the threaded rod 610 of the piston assembly 600, said through-cavity does not have an inner helical rib in the extension of the inner helical rib of the longitudinal internal channel 613.

It is also conceivable that the elongate body of the rod 410 of the mixing assembly 400 has an outer helical rib, i.e. a helical rib formed on an outer surface of the elongate body. The longitudinal internal channel 613 of the threaded rod 610 of the piston assembly 600 has an inner helical groove.

The handle 450 of the mixing assembly 400 is connected to the rod 410 of the mixing assembly 400. A connecting member between the handle 450 and the rod 410 of the mixing assembly 400 allows the rod 410 of the mixing assembly 400 to rotate about the longitudinal axis Z relative to the handle 450.

In this second version, the operator then needs to impart only a translational motion, along the longitudinal axis Z, to the handle 450 of the mixing assembly 400 in order to translate and rotate the mixing paddle 430 of the mixing assembly 400. The engagement of the helical groove of the rod 410 in the helical rib of the longitudinal internal channel 613 of the threaded rod 610 translates and rotates said rod 410, and consequently translates and rotates the mixing paddle 430 in the vessel 200.

In a third version (not shown) of the device 100, the rod 410 of the mixing assembly 400 is an elongate body, having an outer helical groove. The longitudinal internal channel 613 of the threaded rod 610 of the piston assembly 600 has a smooth appearance and has a cross-section larger than the cross-section of the rod 410 of the mixing assembly 400. The through-cavity in the handle 650 of the piston assembly 600 has a cross-section smaller than the cross-section of the longitudinal internal channel 613 of the threaded rod 610 of the piston assembly 600, and has an inner helical rib. The outer helical groove of the rod 410 of the mixing assembly 400 cooperates with the inner helical rib of the through-cavity of the handle 650 of the piston assembly.

The handle 450 of the mixing assembly 400 is connected to the rod 410 of the mixing assembly 400. A connecting member between the handle 450 and the rod 410 of the mixing assembly 400 allows the rod 410 of the mixing assembly 400 to rotate about the longitudinal axis Z relative to the handle 450 of the mixing assembly 400.

In this third version, the operator then needs to impart only a translational motion, along the longitudinal axis Z, to the handle 450 of the mixing assembly 400 in order to translate and rotate the mixing paddle 430 of the mixing assembly 400. Engaging the helical groove of the rod 410 in the helical rib of the through-cavity of the handle 650 of the piston assembly 600 translates and rotates the rod 410 of the mixing assembly 400, and consequently translates and rotates the mixing paddle 430 in the vessel 200.

In one embodiment of the invention, the device 100 can comprise a resilient return member 700 configured to return the mixing assembly to its initial position after each actuation. Preferably, the return member is configured to exert a force on the handle 450 of the mixing assembly 400 so as to bring the mixing assembly back to its initial position.

In a preferred embodiment, the resilient return member is a spring 700.

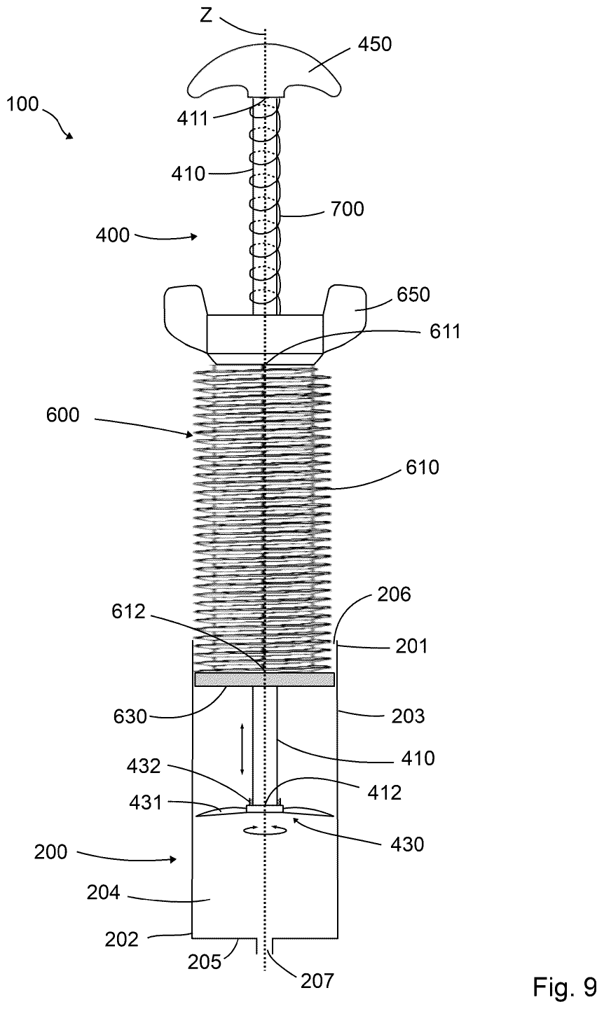

In an example embodiment, as illustrated in FIG. 9, the spring 700 is a helical compression spring. Said helical compression spring is arranged around the rod 410 of the mixing assembly 400, between the handle 450 of said mixing assembly and the handle 650 of the piston assembly 600. When the mixing assembly 400 is in the initial position, the helical compression spring 700 is in the rest position, i.e., it is not compressed.

Thus, in operation, the operator grasps the handle 450 of the mixing assembly 400, which is in the initial position, and imparts a translational, and optionally rotational, motion, depending on the version of the device 100, to the mixing assembly 400 in order to translate and rotate the mixing paddle 430 in the inner chamber 204 of the vessel 200, from the first end 210 towards the second end 220 of said vessel. When the mixing assembly 400 is actuated, the helical compression spring 700 compresses. The operator can then release the handle 450 of the mixing assembly 400 and the mixing assembly automatically resumes its initial position. In other words, after actuating the mixing assembly 400, the compressed helical compression spring 700 will resiliently return to its rest position, driving therewith the mixing assembly into its initial position. To return to its initial position, the rod 410 of the mixing assembly 400 follows a reverse translational motion, and optionally a reverse rotational motion, depending on the version of the device 100. The operator grasps the handle 450 of the mixing assembly 400 again to re-actuate the mixing assembly 400 until the mixing phase is complete.

In another example embodiment (not shown in the figures), the spring 700 is a helical tension spring. Said spring is housed in the longitudinal internal channel 613 of the threaded rod 610 of the piston assembly 600, and arranged around the rod 410 of the mixing assembly 400. This embodiment is applicable only for the third version of the device 100, the longitudinal internal channel 613 of the threaded rod 610 of the piston assembly 600 having a cross-section larger than the cross-section of the rod 410 of the mixing assembly 400, thereby defining a passage enabling the insertion of the spring 700. One of the ends of the spring 700 can, for example, be held rigidly to the first longitudinal end 611 of the threaded rod 610, and the other end of the spring 700 can, for example, be held rigidly to the mixing paddle 430 of the mixing assembly. When the mixing assembly 400 is in the initial position, the helical tension spring 700 is in the rest position, i.e., not extended.

Thus, in operation, the operator grasps the handle 450 of the mixing assembly 400, which is in the initial position, and imparts a translational motion to the mixing assembly 400 in order to translate and rotate the mixing paddle 430 in the inner chamber 204 of the vessel 200, from the first end 210 towards the second end 220 of said vessel. When the mixing assembly 400 is actuated, the helical tension spring 700 extends. The operator can then release the handle 450 of the mixing assembly 400 and the mixing assembly automatically resumes its initial position. In other words, after actuation of the mixing assembly, the stretched helical tension spring 700 will resiliently return to its rest position, driving therewith the mixing assembly into its initial position. To return to its initial position, the rod 410 of the mixing assembly 400 will follow a reverse translational and reverse rotational motion. The operator grasps the handle 450 of the mixing assembly 400 again to re-actuate the mixing assembly 400 until the mixing phase is complete.

Advantageously, the use of a resilient return member 700 allows reducing the repetitive back-and-forth movements of the mixing assembly 400 for the operator, which can prevent or limit musculoskeletal disorders (known under the acronym MSDs).

In one embodiment of the invention, to prevent the mixing paddle 430 from moving in the inner chamber 204 of the vessel 200 during the injection phase, the mixing paddle 430 can be attached to the piston head 630. The mixing paddle is preferably fastened to the piston head so as to simultaneously obstruct the through-hole 632 in the piston head 630.

In a preferred embodiment, the mixing paddle 430 can be attached to the piston head 630 by interlocking, resilient snap-fitting, or screwing.

In the non-limiting example shown in FIGS. 5, 6 and 8, the mixing paddle 430 has a sleeve 432 extending axially, and the piston head 630 has a recess 634 capable of receiving the sleeve 432 by interlocking therewith. The recess 634 has a shape and dimensions that are adapted for rigidly interlocking with the sleeve 432. In the example illustrated in FIGS. 5, 6 and 8, the recess 634 in the piston head 630 is coaxial with the through-hole 632 in the piston head 630 and is of larger dimension than the through-hole 632. The recess 634 can, for example, have a square shape, as illustrated in FIGS. 5 and 6. The sleeve 432 of the mixing paddle 430 is a central sleeve 432 whose external shape complements the shape of the recess 634 in the piston head 630. The blades 431 of the mixing paddle 430 are rigidly connected to the sleeve 432.

To attach the mixing paddle 430 to the piston head 630 by interlocking, the operator must simply pull on the rod 410 of the mixing assembly 400 until the sleeve 432 engages in the recess 634 and becomes interlocked therewith. Thus, the sleeve of the mixing paddle simultaneously closes off the through-hole 632 in the piston head 630.

In one embodiment of the invention, to prevent the rod 410 of the mixing assembly 400 from obstructing the movements of the operator when in the injection phase, the rod 410 of the mixing assembly 400 can be removable.

In one example embodiment, the rod 410 of the mixing assembly 400 can be removed at the second longitudinal end 412.

In one embodiment, as illustrated in FIG. 8, the rod 410 of the mixing assembly 400 comprises, at its second longitudinal end 412, a thread 413 and the sleeve 432 of the mixing paddle 430 comprises a tapped central cavity 433. The thread 413 of the rod 410 and the tapping of the central cavity 433 cooperate with one another. When the operator wishes to remove the rod 410 from the device 100, the operator must have, in advance, attached the mixing paddle 430 to the piston head 630, rigidly connecting the mixing paddle 430 to the piston head 630. The operator can then unscrew the rod 410. When the thread 413 of the rod 410 is out of the tapping of the central cavity 433 of the sleeve 432, the operator can extract the rod 410 from the longitudinal internal channel 613 of the threaded rod 610 of the piston assembly 600.

Alternatively, to prevent the rod 410 of the mixing assembly 400 from obstructing the movements of the operator when in the injection phase, the rod 410 can be broken.

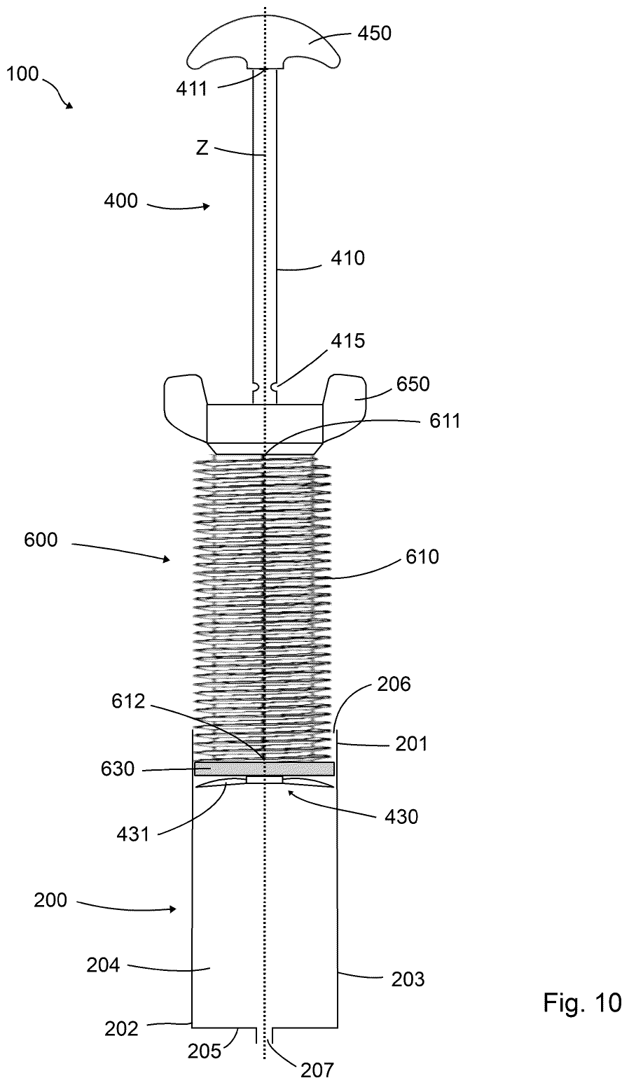

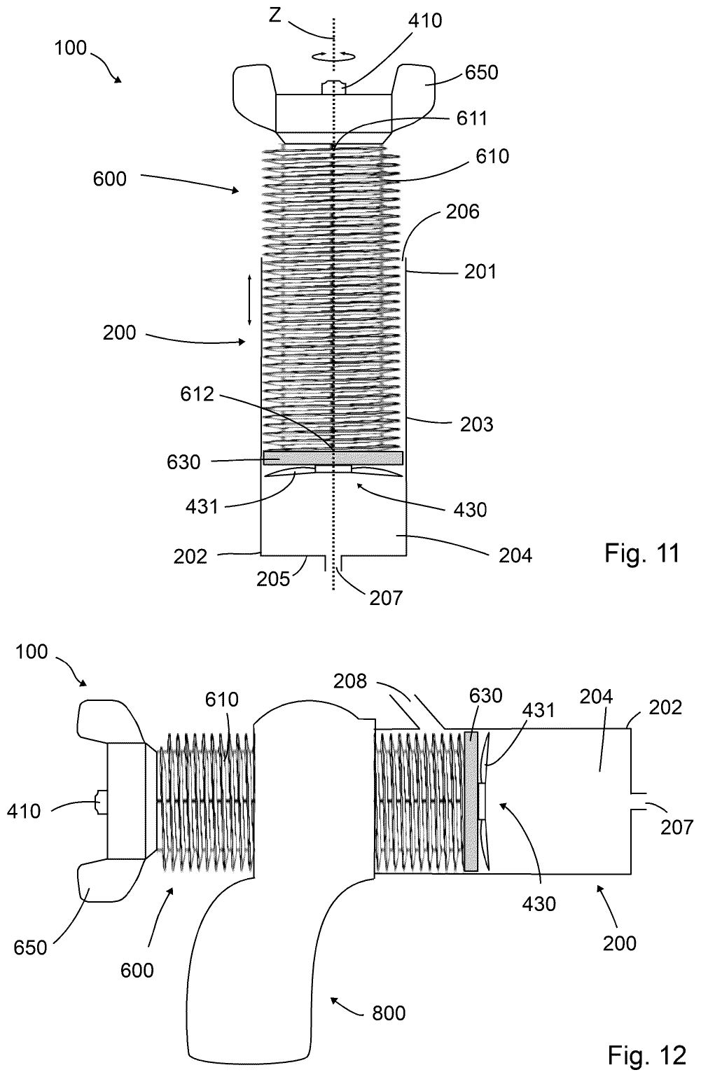

In an example embodiment, as illustrated in FIG. 10, the rod 410 of the mixing assembly 400 can have a weak zone 415 that allows said rod to be broken.

A weak zone is understood to mean a mechanically weaker zone of the rod 410.

In one embodiment, as illustrated in FIG. 10, the weak zone 415 can be a zone of reduced thickness.

In another embodiment (not shown), the weak zone can be a notch.

Preferably, as illustrated in FIG. 10, the weak zone 415 is made on the rod 410, on a portion located at the handle 650 of the piston assembly 600, when the piston head 630 and the mixing paddle 430 are both at the first end 201 of the vessel 200.

The weak zone 415 is preferably broken by a manual action by the operator.

In one embodiment of the invention, illustrated in FIG. 12, to facilitate the operation of the device 100 by the operator, said device comprises a gripping member 800, of the handle type.

In one embodiment of the gripping member (not shown in the figures), said gripping member is formed by two half-shells assembled to one another, enclosing the vessel 200 at its cylindrical wall 203. The first and second ends 201, 202 of the vessel 200 are not enclosed by the two half shells. In this embodiment, the components are introduced either through the hole 208 in the cylindrical wall 203, or through the first end 201 of the vessel 200, when the vessel is made of two removable parts 210, 220, as illustrated in FIG. 2.

In another embodiment, illustrated by FIG. 12, said gripping member is formed by two half-shells assembled to each other, enclosing the vessel 200, at at least its first end 201, and a portion of the threaded rod 610 of the piston assembly 600. Each half-shell can comprise an indentation which, when the two half-shells are assembled, forms a tapping cooperating with the thread of the threaded rod 610 of the piston assembly 600. In this other embodiment, the components are preferably introduced through the hole 208 in the cylindrical wall 203, as illustrated in FIG. 12.

### Operation of the Device 100

One example of the operation of the device 100 will now be described.

In a preliminary phase, the components are introduced into the inner chamber 204 of the vessel.

The piston head 630 of the piston assembly 600 and the mixing paddle 430 of the mixing assembly 400 are each placed toward the first end 201 of the vessel 200.

In one example implementation, when the vessel 200 comprises two removable parts 210, 220, as illustrated in view a) of FIG. 2, the operator disassembles the two parts 210, 220 by unscrewing. Then the operator introduces the components into the second part 220, then assembles the two parts 210, 220 again to close the vessel 200, by screwing the two parts back together.

In another example implementation, when the vessel 200 comprises, at its cylindrical wall 203, a hole 208, as illustrated in view b) of FIG. 2 and views a) and b) of FIG. 3, the operator introduces the components through this hole 208.

Once the components have been introduced into the inner chamber 204 of the vessel, the mixing phase can begin.

The operator grasps the handle 450 of the mixing assembly 400 and imparts a translational motion, and optionally rotational motion, depending on the version of the device 100, to the mixing assembly 400 in order to translate and rotate the mixing paddle 430 in the inner chamber 204 of the vessel 200, between the first end 210 and the second end 220 of said vessel, as illustrated in FIGS. 1 and 9. Moving the mixing paddle 430 into the inner chamber 204 of the vessel 200 thus makes it possible to mix the components with each other until the bone cement is formed.

The piston head 630 of the piston assembly 600 remains in position, at the first end 201 of the vessel 200, during the entire mixing phase.

When the mixing phase is completed, the operator brings the mixing paddle 430 back to the first end 201 of the vessel 200 and attaches the mixing paddle 430 to the piston head 630 of the piston assembly 600 to rigidly connect them.

In one example implementation, the operator pulls on the handle 450 of the mixing assembly 400 until the sleeve 432 of the mixing paddle 430 interlocks inside the recess 634 of the piston head 630 of the piston assembly 600, as illustrated in FIG. 10.

When the mixing paddle 430 is rigidly connected to the piston head 630 of the piston assembly 600, the operator can, depending on the version of the device, either break the rod 410 of the mixing assembly 400, at its weak zone 415, or unscrew the rod 410 of the mixing assembly 400 from the sleeve 432 of the mixing paddle 430.

The injection phase can then begin. The operator grasps the handle 650 of the piston assembly 600 and rotates it clockwise to screw the threaded rod into the tapped opening of the vessel, causing the piston head 630 and the mixing paddle 430 to translate towards the second end 202 of the vessel 200, pushing the obtained bone cement through the outlet 207 of the vessel 200.

Thanks to the engagement of the threaded rod 630 of the piston assembly 600 in the tapped opening 206 of the vessel 200, the device 100 advantageously makes it possible to easily inject the bone cement, even when it has a high viscosity, without breaking.

The description hereinbefore clearly illustrates that, thanks to its different features and their advantages, the present invention achieves the set objectives. In particular, it provides a device that allows mixing the components in order to obtain a composition, as well as injecting it, under high pressure, without excessive handling by the operator.

Although the invention has been described with regard to use in the medical field, use thereof in other fields of application is not in any way excluded. Thus, the device can conceivably be used in the construction sector for the repair of cracks.

1-9. (canceled)

10. A device to mix and inject a composition, comprising:
a vessel comprising a cylindrical wall, a first end and a second end, the vessel defining an inner chamber and having a longitudinal axis, the vessel further comprising a tapped opening at the first end and an outlet at the second end;
a piston assembly comprising a threaded rod, a handle located at a first end of the threaded rod and a piston head located at a second end of the threaded rod;
the threaded rod cooperating with the tapped opening of the vessel, the thread rod extending along the longitudinal axis of the vessel, and the threaded rod comprising two longitudinal ends and a longitudinal internal channel between the two longitudinal ends;
the piston head being arranged in the inner chamber of the vessel, the piston head comprising a cross-section whose shape is complementary to that of a cross-section of the cylindrical wall of the vessel, the piston head comprising a through-hole in a continuation of the longitudinal internal channel of the threaded rod;
the handle of the piston assembly comprising a through-cavity in an extension of the longitudinal internal channel of the threaded rod;
a mixing assembly comprising a rod, a handle located at a first end of the rod, and a mixing paddle located at a second end of the rod, the rod of the mixing assembly extending into the longitudinal internal channel of the threaded rod and into the through-hole of the piston head of the piston assembly, the mixing paddle being arranged in the inner chamber of the vessel;
wherein the rod of the mixing assembly is an elongate body comprising an outer helical groove; and
wherein the longitudinal internal channel of the threaded rod of the piston assembly comprises an inner helical rib, the outer helical groove cooperating with the inner helical rib of the longitudinal internal channel of the threaded rod of the piston assembly.

11. The device of claim 10, wherein the vessel is made of two removable parts, a first part comprising the tapped opening of the vessel and a second part comprising the outlet of the vessel, the first part forming a cover for the second part.

12. The device of claim 10, wherein the vessel comprises a hole made in the cylindrical wall to introduce constituent components of the composition into the inner chamber of the vessel.

13. The device of claim 10, wherein the mixing paddle of the mixing assembly is rigidly connected to the piston head, during an injection phase.

14. The device of claim 13, wherein the mixing paddle of the mixing assembly comprises a sleeve, and the piston head of the piston assembly comprises a recess configured to receive the sleeve by interlocking therewith.

15. The device of claim 10, wherein the rod of the mixing assembly is removable.

16. The device of claim 10, wherein the rod of the mixing assembly comprises a weak zone to allow the rod to break.

17. The device of claim 10, further comprising a gripping member.

18. The device of claim 10, further comprising a resilient return member configured to return the mixing assembly to an initial position in which the mixing paddle of the mixing assembly is against the piston head of the piston assembly.

19. Device to mix and inject a composition, comprising:
a vessel comprising a cylindrical wall, a first end and a second end, the vessel defining an inner chamber and having a longitudinal axis, the vessel further comprising a tapped opening at the first end and an outlet at the second end;
a piston assembly comprising a threaded rod, a handle located at a first end of the threaded rod and a piston head located at a second end of the threaded rod;
the threaded rod cooperating with the tapped opening of the vessel, the thread rod extending along the longitudinal axis of the vessel, and the threaded rod comprising two longitudinal ends and a longitudinal internal channel between the two longitudinal ends;
the piston head being arranged in the inner chamber of the vessel, the piston head comprising a cross-section whose shape is complementary to that of a cross-section of the cylindrical wall of the vessel, the piston head comprising a through-hole in a continuation of the longitudinal internal channel of the threaded rod;
the handle of the piston assembly comprising a through-cavity in an extension of the longitudinal internal channel of the threaded rod;
a mixing assembly comprising a rod, a handle located at a first end of the rod, and a mixing paddle located at a second end of the rod, the rod of the mixing assembly extending into the longitudinal internal channel of the threaded rod and into the through-hole of the piston head of the piston assembly, the mixing paddle being arranged in the inner chamber of the vessel;
wherein the rod of the mixing assembly is an elongate body comprising an outer helical groove; and
wherein the longitudinal internal channel of the threaded rod of the piston assembly has a smooth appearance and a cross-section larger than a cross-section of the rod of the mixing assembly, the through-cavity of the handle of the piston assembly has a cross-section smaller than the cross-section of the longitudinal internal channel of the threaded rod of the piston assembly and comprises an inner helical rib, the outer helical groove cooperating with the inner helical rib of the through-cavity of the handle of the piston assembly.

20. The device of claim 19, wherein the vessel is made of two removable parts, a first part comprising the tapped opening of the vessel and a second part comprising the outlet of the vessel, the first part forming a cover for the second part.

21. The device of claim 19, wherein the vessel comprises a hole made in the cylindrical wall to introduce constituent components of the composition into the inner chamber of the vessel.

22. The device of claim 19, wherein the mixing paddle of the mixing assembly is rigidly connected to the piston head, during an injection phase.

23. The device of claim 22, wherein the mixing paddle of the mixing assembly comprises a sleeve, and the piston head of the piston assembly comprises a recess configured to receive the sleeve by interlocking therewith.

24. The device of claim 19, wherein the rod of the mixing assembly is removable.

25. The device of claim 19, wherein the rod of the mixing assembly comprises a weak zone to allow the rod to break.

26. The device of claim 19, further comprising a gripping member.

27. The device of claim 19, further comprising a resilient return member configured to return the mixing assembly to an initial position in which the mixing paddle of the mixing assembly is against the piston head of the piston assembly.
